(19) United States
(12) Patent Application Publication
(10) Pub. No.: US 2018/0055720 A1
(43) Pub. Date: Mar. 1, 2018

(54) Electrodynamic methood of massage and massage device

(71) Applicants: Lev N. Sitkovetskiy, New London, CT (US); Dmitiy L. Sitkovetskiy, East Lyme, CT (US); Daniel D. Sitkovetskiy, East Lyme, CT (US)

(72) Inventors: Lev N. Sitkovetskiy, New London, CT (US); Dmitiy L. Sitkovetskiy, East Lyme, CT (US); Daniel D. Sitkovetskiy, East Lyme, CT (US)

(21) Appl. No.: 15/248,359

(22) Filed: Aug. 26, 2016

(51) Int. Cl. A61H 7/00; A61N 1/20

(52) U.S. Cl. CPC A61H 7/004; A61N 1/205; A61H 2201/1207; A61H 2209/00; A61H 2201/10

(57) ABSTRACT

Electrodynamic massage device comprises of a constantly reversible motion mechanism, an electrical engine or electromagnet, and a moving shaft with a changeable influencing head executed or covered on its open end with material having electrostatic capabilities, and the motion mechanism is located in the housing of the massage device, and electrical engine or electromagnet are attached to housing. Electrodynamic massage device can perform the massage which creates an electrostatic current on the skin's surface and constant reverse movements of the skin and subcutaneous tissue with purpose of treatment.

### FIELD OF THE INVENTION

The present invention is based on application Ser. No. 13/463,744.

The present invention generally relates to the massage methods and electric massage devices for applying a micro current and friction with pressure to movement on the patient's skin with the objective of improvement for the bodies condition as well as diagnosis, prevention and treatment of diseases.

### BACKGROUND OF THE INVENTION

Many existent methods of massage and massage devices have been proposed or made to supply influence by friction with pressure by massage to the patients. Some stimulation devices and according methods have been proposed or made to supply electrical current for the treatment of organs or nerves. However, none of them are specifically directed to the need of the method and massage device with complex opportunity for improvement of the bodies condition as well as diagnosis, prevention and treatment of diseases.

Methods of diagnosis related to massage are palpation and methods that use instruments for substitution palpation. The most pertinent prior arts known to the inventions are U.S. Pat. No. 6,887,211 for through soft tissue mobilization therapies. Some massage instruments also have additional properties for increasing their influence. U.S. Pat. No. 5,843,005 contains a negative ion emitting surface covering at least a portion of the tool part or the handle, and the negative ion emitting surfaces consist of ebonite.

All known methods of the instrument's massage have limited possibilities and do not reach efficiency of the diagnosis with palpation and manual massage of the classical system. Accordingly, the instrument massage devices have limited use in the treatment and improvement of the bodis condition.

Some methods of physical therapy consist of creating a stimulation effect by supplying electrical current to treatment organs or nerves. The most pertinent prior arts known to the inventions are: U.S. Pat. No. 6,443,915 B1 consists of beautifying face skin by galvanic ion current, infrared ray, and vibration, and measuring the skin state, U.S. Pat. No. 8,784,460 B2 consists of micro current delivery device portion with a sensory cue, U.S. Pat. No. 8,798,739 B2 consists of a therapeutic electrical stimulation device includes a shoe delivered electrical current from controller, through the conductor and the electrodes.

All known methods of stimulation and stimulation devices have limited possibilities only for treatment and can not be used as massage devices.

The methods of massage therapy consist of creating friction between the skin of patient and an influencing part of a massage device with pressure. There are many types of massage methods and electric massage devices related to the type of moving influencing parts of massage device and design of this one. The most pertinent prior arts known to the inventions are:

U.S. Pat. No. 9,289,348 B2 where massage head simulates human physical contact by moving back and forth, U.S. Pat. No. 8,951,216 where beating member reciprocates back and forth by rotating shaft skin massage device of rubbing a skin, U.S. Pat. No. 8,777,881 where the basic plate moves back and forth along of an axle of the motor, providing kneading and percussion massage of the users body, U.S. Pat. No. 6,994 where a massage device capable of producing vibration or reciprocating motions for massaging muscles of a person, U.S. Pat. No. 6,517,499 contains the rollers with reciprocating linear motion, U.S. Pat. No. 6,251,089 contains massage heads to obtain selected stroking, rubbing and/or kneading massaging effects by an active or passive reciprocal arc movement for body and joints massage, U.S. Pat. No. 5,085,207 contains a finger, which is powered in a back and forth transverse motion, and CN Pat. 201658505 wherein a middle pin shaft is driven to realize back-and-forth swinging, thus leading the massage head to simulate a massage method and a kneading method of the traditional Chinese medicine massage.

All known methods of massage and electric massage devices are efficient enough for the influence upon soft tissue and muscular processes and diseases connected with these processes, but insufficient for increasing the energy metabolism of the organism, strengthening the muscles and capillary blood circulation, improving appearance of persons skin, and reduction of the inflammation processes. They do not have high level of the influence upon skin and subcutaneous processes as well as the influences upon tissue swelling and bone outgrowth, and, moreover, do not have the possibility of diagnosis and prevention of diseases.

The novel method of massage and the massage device is intended for eliminating this deficiency. Proposed novel method of the massage and the massage device solves the specified problems above, and vastly exceeds the efficiency of the known massage devices and the manual massage methods. This result was reached by the principal new massage method of influence on the skin and internal tissue.

### SUMMARY OF THE INVENTION

The present invention was designed with the objective of creating a scientifically motivated massage method for the improvement of the body condition as well as diagnosis, prevention and treatment of numerous diseases by using the novelty physical method of massage on the bodies surfaces with the highest level of the efficiency, and development of the possible massage devices corresponding to these purposes.

The most essential differences of the novel invention are that the electrodynamic method of massage for improvement of the bodies condition, diagnosis, prevention and treatment of diseases consists of creating an electrostatic current on the skins surface and creating increased blood circulation under the skin and subcutaneous tissue by constant reverse movements of skin and subcutaneous tissue including linear reciprocating or constant reversible angular turning movements with or without sliding of the skin under influence with friction of constant reverse movements including linear reciprocating or reversible angular turn movements of influencing head of the massage device having electrostatic capabilities, and the constant or variable pressure applied by the massage device to the skin's surface, and stop the massage device with the movements of the skin by the influencing head in the diagnostic points with pain or treatment points, or the movements of the massage device and the influencing head along the patient's skin surface in the treatment or in the improvement of the bodies condition zones.

The electrodynamic method of massage for the improvement of the body condition, diagnosis, prevention and treatment of diseases contains two types of movements of massage device on the skin surface. One type is the movement of the massage device under pressure created by user, and second type is the constant movement of influencing head of the massage device created by the motion mechanism of massage device.

The friction between the skin of the patient and the friction end of constant working influencing head of the massage device having electrostatic capabilities creates the electrostatic current on the skin's surface and a pumping effect of increasing the blood circulation is produced. The electrostatic current applied on the body's surfaces has treatment properties including the treatment of inflammatory diseases, and this one has pain relief effects too.

The pumping effect of increasing the blood circulation has a high level of treatment of a lot of diseases, and this one has pain relief effects too. According to our research the electrostatic current on the skin surface boosts effectiveness of the pumping effect of increasing blood circulation and vice versa.

The constant reverse movements including linear reciprocating or reversible angular turning movements of influencing head of the massage device works in next manner. When the influencing head start and reverse, the speed of this one is zero. In this time the electrostatic current is absent. When the influencing head slides along the skin a small electrostatic current is produced. The movements of the influencing head can only produce small electrostatic current while interrupting it in the time of start and reverse. This type of electrostatic current is like a micro direct current. This novelty complex type of the method of massage, which create the electrostatic current and pumping effect of increasing blood circulation by constant reverse movements of the skin and subcutaneous tissue, was named as electrodynamic method of massage.

When the influencing head does not slide along the skin the electrostatic current is not produced. This gives the opportunity to the operator of the massage device to create some next regimes or combination of them.

Regime of creating electrostatic current and small pumping effect.

The operator of the massage device uses small pressure on the skin surface. The constantly working influencing head most time slides along the skin, and the electrostatic current and small pumping effect are produced.
This regime is good for patients with skin problems.

Regime of mixed electrostatic current and increasing blood circulation by reversing skin surface.

The operator of massage device uses middle pressure on the skin's surface. The constantly working influencing head partly slides along the skin and partly moves the skin without sliding, and the small electrostatic current with increasing of blood circulation is produced.
This regime is universal for all proposal of electrodynamic massage.

Regime of increasing blood circulation by most time reversing skin surface and producing small electrostatic current only in the time of reverse.

The operator of the massage device uses high pressure on the skin's surface. The constantly working influencing head most time does not slide along the skin, this one moves with reversing the skin's surface and subcutaneous tissue most time without sliding, and increasing blood circulation and small electrostatic current are produced.
This regime is good for diagnosing diseases and for pain relief.

According to the present invention, the electrodynamic method of improvement of the body condition as well as diagnosis, prevention and treatment of diseases can consist of creating the electrostatic current on skin's surface and increasing blood circulation by constant reverse movements with or without sliding on the skin under friction of constant working reverse movements of influencing head of the massage device with constant or variable pressure applied to the skin's surface and stopping the massage device with the movement of the skin in the diagnostic points with pain, and waiting for the patient's response to pain for some time, but for no more than two minutes. If the pain does not disappear, continue the said movements of the said massage device applied in the area of the said diagnostic point with pain until the pain disappears, but for no more than ten minutes.

If the pain does not disappear in this time, the patient needs a more serious treatment.
This type of massage creates electrostatic current on the skin's surface and involves constant reverse movement on the skin with subcutaneous tissue and vessels and as result the pumping effect of increasing of blood circulation is done.

The appearance of pain in the diagnostic points tells of first step of disease, when the patient does not feel himself as ill, or of the disease in the patient's organ in this area. The disappearance of pain under the electrodynamic method of massage tells the possibility of the prevention of disease, or of the result of treatment.

According to the present invention, the electrodynamic method of improvement of the bodies condition as well as diagnosis, prevention and treatment diseases can consist of creating the electrostatic current on the skin's surface and the increasing of blood circulation by constant reverse movements with or without sliding of the skin under friction of constant working reverse movements of influencing head of the massage device with the constant or variable pressure applied to the skin's surface, and checking the presence of the skin showing of red, and stop the said massage device in one point for no more than of two minutes or repeat the said movements of the said massage device in the treatment areas of the body for no more than ten minutes until the treatment effect is done by increasing the skin reddening.

The time of increasing the skin reddening tells of level of disease and the result of treatment. Then more this time, then more the stage of disease. This time can has a good use as the method of diagnosis, prevention and treatment of diseases.

The named above procedures have to repeat some session until the pain fully disappears or the treatment effect or improvement of body's condition will be done.

The possibilities of the novel electrodynamic massage method exceed the possibilities of manual and known methods of massage in improving the body's condition and the treatment of numerous diseases.

Moreover, the novel electrodynamic massage method can be used for stimulation of some exchange processes, improvement of peripheral blood circulation, increasing the body serviceability, elimination of pain syndrome, improvement of cartilaginous tissue, elimination of tendons disorders, subcutaneous condensation and bones outgrowths, treatment of some inflammation processes etc.

The novel electrodynamic massage method can have good use in the physical therapy, sport medicine, for a cosmetic purpose, and in everyday life. The treatment with the proposed massage method can be combined with many known methods of treatment, including pharmacology, herbs and physiotherapy. This method intensifies their activity in the process of such treatment in the majority of cases.

Many types of the massage devices can be designed for the method of diagnosis, prevention and treatment of diseases, and improvement of body condition. Some types of design and their drawing of the massage devices are presented in this invention as examples.

According to the present invention, the main principle of electrodynamic massage device designs comprises a constantly working reversible motion mechanism, including linear reciprocating or reversible angular turning motion mechanisms, and the motion mechanism contains an electrical engine or electromagnet, and the motion mechanism contains a moving shaft, and the moving shaft contains on the open end the changeable influencing head, and the influencing head executed or covered on the open end with material having electrostatic capabilities, and the open end of the influencing head executed in the form of friction surface flat or spherical form, and the motion mechanism is located in the housing of the massage device, and the electrical engine or electromagnet is attached to the housing.

According to the present invention, the housing of the massage device can be executed in the form of a pistol.

According to the present invention, the motion mechanism contains the constantly working electromechanical system of reverse, and the system of reverse consists of the electrical motor with the moving shaft, a spring, two levers, and two switches, and one switch is main switch with normally opens lug terminals, and the second switch is an operating switch with normal closet lug terminals, and the two switches and spring are attached to the housing, and the two levers are attached to the moving shaft, and the electric cord of the source of current is attached to the electrical motor via the switches.

According to the present invention, the motion mechanism contains the constantly working electrical system of reverse, and the system of reverse is the reversible direct current electrical motor with the moving shaft and capacitor operated by alternative current, and the cord of the source of alternative current is attached via the switch to the capacitor and electrical motor.

Because the proposed electrodynamic method of massage is a novelty, a lot of massages design can be created for this method, the most of them related to the system of reverse.

For instance, electromechanical system of reverse with the electromagnet is presented.
The motion mechanism contains the constantly working electromechanical system of reverse, and the system of reverse contains the electromagnet, the electromagnet consists a continuous push/pull solenoid with spring, the moving shaft is the shaft of the solenoid, the moving shaft contains an changeable influencing head, the influencing head executed or covered on the open end with material having electrostatic capabilities, and two switches with normally open lug terminals, and one switch is the main switch, and the second switch is the operating switch, and the cord of the source of current is attached via the main switch to the operated switch and the solenoid etc.

The changing of the power parameters from the electric source to the a constantly working reverse motion mechanism can be produced by the transformer.

According to the present invention, the friction surface of the influencing head of the massage device executed or covered on the open end with material having electrostatic capabilities, and these materials are ebonite or amber.

According to the present invention, the friction surface of the influencing head of the massage device contain useful substances such as medicine or anti-inflammatory metals such as copper or silver.

### BRIEF DESCRIPTION OF THE DRAWINGS

For more detailed explanation and understanding of the persisting invention by description of the method and examples of design of the massage device below present drawings, which is an explanation to the figures:

FIG. 1 presents the perspective view of the massage device with the electrical motor and the electromechanical system of reverse, and the cylindrical form housing with section views of main parts designs of the massage device.

FIG. 2 present the perspective view of the moving parts of the massage device with the electric engine and the electromechanical systems of reverse.

FIG. 3 presents the perspective view of the massage device with the direct current electrical motor and the electrical system of reverse, and the housing of the pistol form with section views of main part's designs of the massage device.

### DETAILED DESCRIPTION OF THE DRAWINGS.

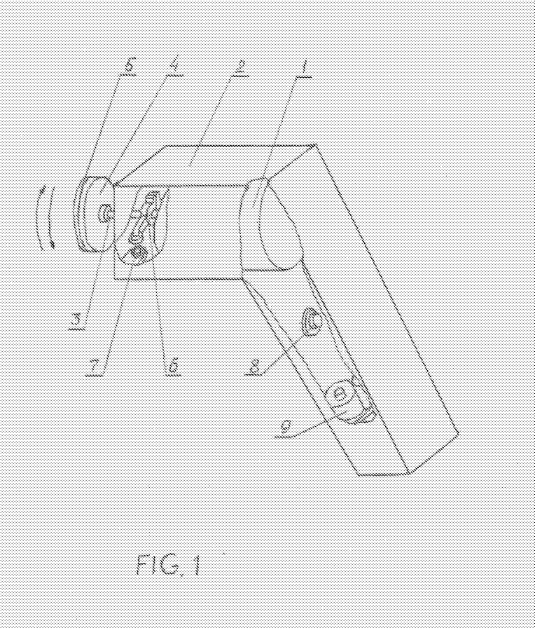

FIG. 1 presents the general view in perspective of the massage device operated by the electro-mechanical system of reverse. The massage device consisting of the electric motor 1 located in the housing 2. The shaft 3 of the electric motor 1 contains the changeable influencing head 4 on the open end and the system for reverse 6 in the middle part. The influencing head 4 covered by material 5 such as ebonite, which creates the electrostatic potential on the skin's surface.

The electromechanical system for reverse 6 consists of two levers 7 and 9. One lever 7 is for applying contact with the operating switch with normally closet lug terminals 8 and the second lever 9 for tensing flat spring 10 for reverse. The housing 2 contains the operating switch with normally closet lug terminals 8, the main switch with normally open lug terminals 11 and the electric battery 12.

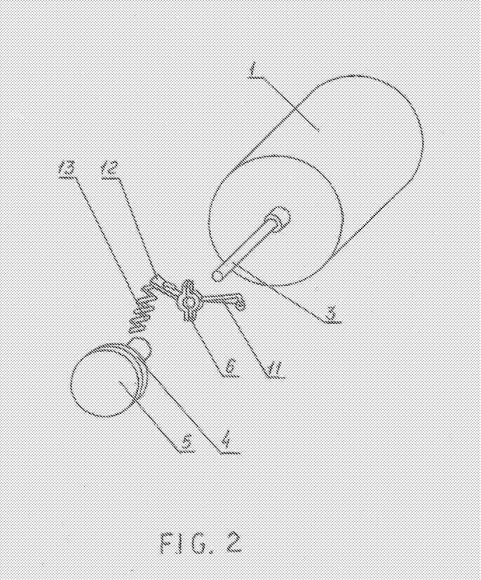

FIG. 2 presents the perspective view of the moving parts of the massage device consisting of the shaft 3 of the electric motor 1. The shaft 3 in the middle part contains the electromechanical system for reverse 6 that consists of two levers. One lever 7 is for applying contact with the operating switch with normally closet lug terminals 8 and the second lever 9 for tensing spring 10 for reverse. The open end of the shaft 3 contains the changeable influencing head 4. The end of the head 4 is covered with material 5, which can create the electrostatic potential on the body surface such as ebonite.

The massage device operated by electromechanical system of reverse with the electric motor 1 works in the next manner: when the main switch 11 is turned on, the electric motor 1 receives the electric current from the electric battery 12 and turns its shaft 3 together with influencing head 4 and both levers 7 and 9 of the system of reverse 6. The contacting leg 7 attracts the operating switch with normally closet lug terminals 8 and turns it off. The electric engine 1 is discharged and its shaft 3 together with the influencing head 4 stops. In the same time, the opposite leg 9 stretches out the spring 10. When the electric engine 1 is discharged, the spring 10 returns the shaft 3 together with influencing head 4 and both legs 7 and 9 of reverse system 6 in the starting position. The operating switch with normally closets lug terminals 11 turns on, and the motor 1 receives the electric current from the electric battery 12 and turns its shaft 3. And the process is repeated. As result we received constantly working reversible angular turning movements of influencing head 4 of massage device. When the shaft 3 turns together with influencing head 4 forward and backward the end of the influencing head 4 under manual pressure applied to the patient's skin surface, this movement creates the friction with the patient's skin under manual pressure. The skin turns forward and backward with sliding under influence of this friction.

When the influencing head slides along the skin the small electrostatic current is produced. The movements of the influencing head can only produce small electrostatic current with interrupting it in the time of start and reverse. This type of electrostatic current is like a micro direct current influence of this friction.
When the influencing head turns forward and backwards on the skin with the forwards and backwards reversible angular turning movement, the skin with subcutaneous tissue and vessels creates the pumping effect of blood circulation. As result, the bloods circulation in the influenced part of the skin increases and the treatment and diagnosis effects are produced.

These possibilities of the massage device give the opportunities of diagnosis, prevention and treatment diseases, and improvement of a body condition in the numerous diseases possible and can have use in the physical therapy, sport medicine, for cosmetic purpose, and in everyday life.

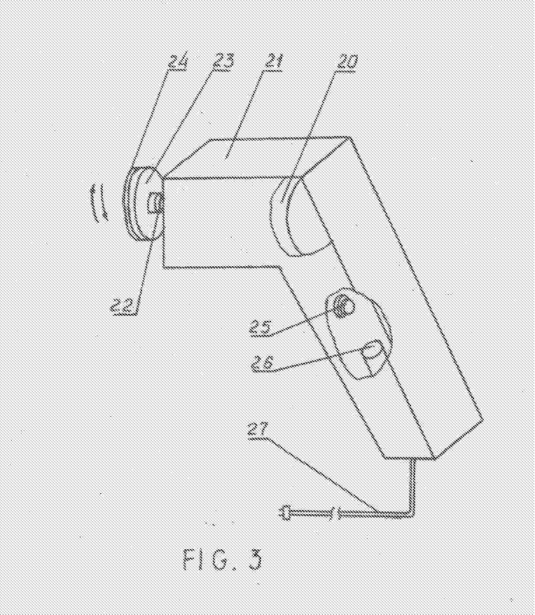

FIG. 3 presents the general view in perspective of the massage device operated by electrical system of reverse. The massage device consists of the electric motor 20 located in the pistol for housing 21. The shaft 22 of the electric motor 20 contains the changeable influencing head 23 on the open end. The influencing head 23 covered with material 24, which creates the electrostatic current on the body surface such as ebonite. The housing 21 contains the switch 25, the capacitor 26, and electric cord 27 for contact with the source of alternative current via standard transformer The transformer is not disposed.

The massage device operated by electromechanical system of reverse has the same possibilities as the massage device operated electrical system of reverse. The forwards and backwards reversible angular turning motion of the influencing head 23 of the electric engine 20 is produced by alternative current.

It should be understood that the preferred embodiment and examples described herein are for illustrative purposes only and are not to be construed as limiting the scope of the present invention which is properly delineated only in the appended claims. It should also be understood that the drawings are not necessarily drawn to scale, but that they are merely conceptual in nature.

We claim:

1. Electrodynamic method of massage consists of the improvement of the bodies condition, diagnosis, prevention and treatment of diseases, said method of massage involves creating electrostatic current on the skin's surface and constant reverse movements of said skin and subcutaneous tissue under friction of constant reverse movements along the skin of an influencing massage head device having electrostatic capabilities, and a constant or variable pressure applied by the massage device to said skin's surface, and to stop or to move said massage device with said constantly reverse movements of the influencing head in the diagnostic points or in the treatment and/or improvement of the bodies condition zones.

2. Electrodynamic massage device comprises of a constantly reversible motion mechanism, and said motion mechanism contains an electrical engine or electromagnet and a moving shaft, said moving shaft contains a changeable influencing head, and said influencing head executed or covered on its open end with material having electrostatic capabilities, and the open end of said influencing head executed in the form of friction surface, and said motion mechanism is located in the housing of the massage device, and said electrical engine or electromagnet are attached to said housing.

3. Electrodynamic massage device according to claim 2 wherein said motion mechanism contains an electro-mechanical constant reverse system, and said reverse system consists of a spring, two levers, and two switches, and one switch is the main switch with normally open lug terminals, and the second switch is the operating switch with normally closets lug terminals, and said two switches and spring are attached to said housing, and said two levers are attached to said moving shaft, and the electric cord of the source of current is attached to said electrical engine via said switches.

4. Electrodynamic massage device according to claim 2 wherein said motion mechanism contains an electrical constant reverse system, said reverse system consists of a reversible direct current electrical motor and capacitor operated by alternative current, and said moving shaft is an axis of said electrical motor, and the end of said axis of said electrical motor contains said changeable influencing head, and the cord of the source of alternative current is attached via the switch to said capacitor and electrical motor.

5. Electrodynamic massage device according to claim 2 wherein the friction surface of said end of the influencing head of the massage device contains useful substance, and said substance consists of medicine.

6. Electrodynamic massage device according to claim 2 wherein the friction surface of said end of the influencing head of the massage device contains useful substances, and said substances consist of the anti-inflammatory metals, and said metals consists of cooper and/or silver.

7. Electrodynamic massage device according to claim 2 wherein said material having electrostatic capabilities contains ebonite or amber.
